SEQUENCE LISTING (3 sequences)

SEQ ID NO: 1 - protein, 23 aa, Artificial Sequence
  Synthetic peptide
IXAVXWAXATAEGGGAGAAHAPG

SEQ ID NO: 2 - protein, 24 aa, Artificial Sequence
  Synthetic peptide
IXAVXWAXPGATAEGGGAGAAHAC

SEQ ID NO: 3 - protein, 25 aa, Artificial Sequence
  Synthetic Peptide
IXAVXWAXPGATAEGGGGAGAAHAA

(19) United States
(12) Patent Application Publication
(10) Pub. No.: US 2012/0053115 A1
(43) Pub. Date: Mar. 1, 2012

(54) Lantibiotic Carboxyamide Derivatives With Enhanced Antibacterial Activity

(75) Inventors: Sonia Ilaria Maffioli, Milano (IT); Cristina Brunati, Monza (MI) (IT); Donatella Potenza, Milano (IT); Francesca Vasile, Milano (IT); Stefano Donadio, Malnate (VA) (IT)

(73) Assignee: Sentinella Pharmaceuticals, Inc., Princeton, NJ (US)

(21) Appl. No.: 13/130,720

(22) PCT Filed: Nov. 24, 2008
(86) PCT No.: PCT/IB2008/003184

§ 371 Date: Nov. 15, 2011

(51) Int. Cl. A61K 38/16; C07K 1/107; A61P 31/04; C07K 14/00

(52) U.S. Cl. 514/2.6; 530/323; 514/2.9; 514/2.7; 514/2.8

(57) ABSTRACT

The present invention relates to novel amide derivatives of the lantibiotic 97518 and their uses. In particular, the present invention describes novel compounds having general formula (II) and their use as antibiotic.

### BACKGROUND OF THE INVENTION

The compounds designated as lantibiotics are peptides characterized by the presence of the amino acids lanthionine and/or 3-methyllanthionine (H. G. Sahl and G. Bierbaum. Lantibiotics: biosynthesis and biological activities of uniquely modified peptides from gram-positive bacteria—Ann. Rev. Microbiol. 52 (1998) 41-79). The teen lantibiotic thus defines a structural feature of these compounds and not necessarily a common possible use. In fact, some lantibiotics possess antibacterial activity while others are totally devoid of it. Among the lantibiotics possessing antibacterial activity, of particular relevance are those active against methicillin-resistant Staphylococcus aureus (MRSA), which can be of considerable interest in medicine. All the lantibiotics endowed with antibacterial activity described so far exert their action by interfering with cell wall biosynthesis, through sequestration of a key intermediate in peptidoglycan formation.

The antibacterial lantibiotics can be broadly divided into two groups on the basis of their structures: type-A lantibiotics are typically elongated, amphiphilic peptides, while type-B lantibiotics are compact and globular (McAuliffe, R. P. Ross and C. Hill. Lantibiotics: structure, biosynthesis and mode of action-FEMS Microb. Rev. 25 (2001) 285-308). Nisin is the typical representative of type A lantibiotic, whereas actagardine and mersacidin belong to the type B lantibiotic subclass. Remarkably, despite differences in shape and primary structure, both nisin-type and mersacidin-type lantibiotics interact with the membrane-bound peptidoglycan precursor lipid II. Furthermore, while the spectrum of antibiotic activity is generally restricted to Gram-positive bacteria, individual members of subclasses A and B greatly vary in their potency. Overall, the structural elements responsible for increased target binding and/or enhanced antibacterial activity in lantibiotics are poorly understood.

Traditionally, lantibiotics have been isolated mostly from the order Firmicutes (low G-C Gram-positive bacteria) and relatively few have been described from the Actinomycetales, the order best known for the ability to produce a large variety of other antibiotics. Actagardine and the recently described 107891 (Patent EP03016306.7; Chemistry and Biology 2008, 15, 22-31) and 97518 (Patent EP14811986A1; Biochemistry 2007, 46, 5884-5895) are representative lantibiotics produced by the Actinomycetales. In particular, the novel lantibiotic 97518 is produced by Planomonospora sp. DSM 14920 and it was found to inhibit cell wall biosynthesis in bacteria (Patent EP14811986A1; Biochemistry 2007, 46, 5884-5895). On the basis of its globular structure, the lantibiotic 97518 was assigned to the mersacidin subgroup of type B lantibiotics (Biochemistry 2007, 46, 5884-5895). 97518 is active in vitro against MRSA, streptococci and enterococci. S. aureus can cause life-threatening infections and MRSA is of particular clinical significance because it is resistant to all penicillins and cephalosporins and also to multiple other antibiotics; in addition it easily spreads from patient to patient causing outbreaks of infection with important implications for healthcare facilities. Vancomycin resistant enterococci (VRE) are emerging as important hospital-acquired pathogens responsible for severe human infections (such as endocarditis, meningitis and septicemia) posing an increasing therapeutic challenge (Y. Cetinkaya, P. Falk and C. G. Mayhall. Vancomycin-resistant enterococci-Clin. Microbiol. Rev. 13 (2000) 686-707; L. B. Rice. Emergence of vancomycin-resistant enterococci. Emerg. Infec. Dis. 7 (2001) 183-7). Streptococcus pneumoniae and Moraxella catarrhalis are recognized important human pathogens. They are a common cause of respiratory tract infections, particularly otitis media in children and lower respiratory tract infections in the eldery. M. catarrhalis and S. pneumoniae have been recently accepted as the commonest pathogens of the respiratory tract (M. C. Enright and H. McKenzy. Moraxella (Branhamella) catarrhalis—Clinical and molecular aspect of a rediscovered pathogen. J. Med. Microbiol. 46 (1997) 360-71). However, despite its interesting antibacterial spectrum, the lantibiotic 97518 has only modest activity against relevant human and animal pathogens, with minimal inhibitory concentrations (MIC) ranging from 0.25 to >128 μg/ml.

Variants and/or derivatives of naturally occurring antibiotics have been long sought after and can be useful in medicine. They can be produced by chemical synthesis or by modification of a natural product, but most structural variations in naturally occurring antibiotics tend to abolish or severely impair their antibacterial activity. This is particularly true in the field of lantibiotics where structure-activity relationships (SAR) are poorly defined, in the absence of molecular details about antibiotic-target interactions. Furthermore, other factors likely to contribute to antibacterial potency are the diffusion rate of the compound to the target, after crossing the thick peptidoglycan layer, and possible interactions with polar, charged and hydrophobic moieties present on the protective external surfaces of the bacterial cell. An additional element rendering unpredictable the outcome of lantibiotic modifications is the existence of unrelated compounds possessing a similar mechanism of action, preventing conclusions drawn from SAR studies on one subtype to be applied to the other.

The structure of the lantibiotic 97518 has been reported as formula (I) (Castiglione et al. 2007) and claimed to belong to lantibiotic subclass B.

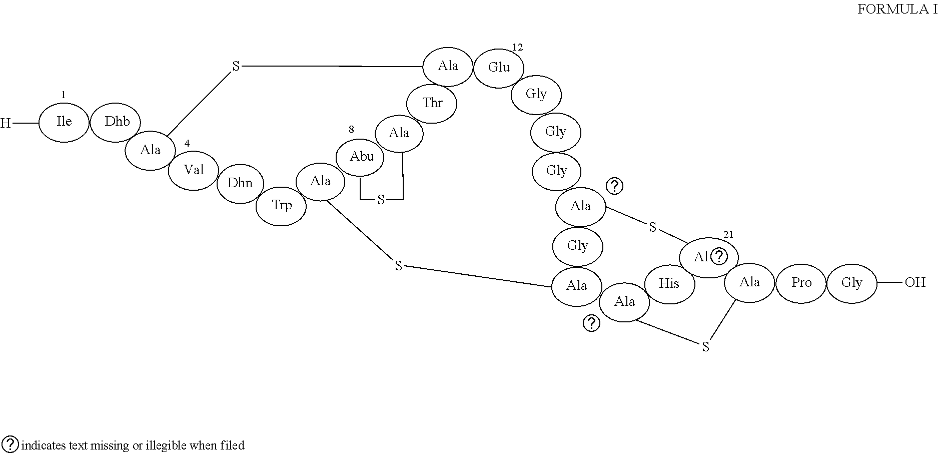

Inspection of the reported primary structure of 97518 indicated similarity to the nisin-type of lantibiotic, which would be enhanced by some thioether linkages. The present invention describes novel derivatives of the lantibiotic 97518 possessing enhanced antibacterial activity having the general formula (II), wherein some of the original structural features reported for 97518 were incorrectly assigned. The novel derivatives have antibacterial activities which are substantially better than that of 97518 itself. The invention thus provides novel antibiotic compounds, methods of making such compounds and their use in the treatment of human or animal subjects, particularly in conditions requiring antibacterial therapy. These and other aspects of the invention are described herein.

### DESCRIPTION OF THE INVENTION

The present invention describes novel antibiotic compounds having the general formula (II)

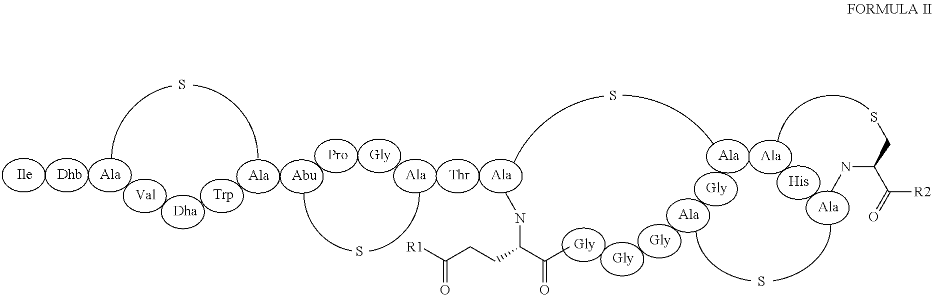

wherein at least one R₁ and R₂ substituent represents the group —NR₃R₄ whereas the other substituent represents either —OH or —NR₃R₄, and wherein R₃ and R₄ independently represent:
hydrogen or
an alkyl of 1 to 20 carbon atoms;
an alkenyl of 2 to 20 carbon atoms;
an alkynyl of 2 to 20 carbon atoms;
a cycloalkyl of 3 to 8 carbon atom optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms;
a phenyl radical optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms
a benzyl radical optionally substituted on the phenyl ring by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms
a naphthyl radical optionally substituted by one or two substituents selected from halo, lower alkyl of 1 to 4 carbon atoms optionally substituted by 1 to 3 halogen atoms, and lower alkoxy of 1 to 4 carbon atoms optionally substituted by 1 to 3 halogen atoms
a group of formula

—(CH₂)ₙOR₅
in which n represents an integer from 2 to 8 and R₅ represent
   hydrogen or
   (C₁-C₄) alkyl or
   a cycloalkyl of 3 to 8 carbon atom optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms.
   a phenyl radical optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms
a group of formula

—(CH₂)ₙNR₆R₇
in which n represents an integer from 2 to 8 and R₆ and R₇ independently represent
   hydrogen or
   (C₁-C₄)alkyl or
   a cycloalkyl of 3 to 8 carbon atom optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms.
   phenyl radical optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms
   a benzyl radical optionally substituted on the phenyl ring by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms
   R₆ and R₇ taken together represent a —(CH₂)₃, —(CH₂)₄—, —(CH₂)₂—O—(CH₂)₂, —(CH₂)₂—S—(CH₂)₂ or
   R₆ and R₇ taken together with the adjacent nitrogen atom represent: a piperazine mojety which may be substituted in position 4 with a substituent selected from (C₁-C₄)alkyl, (C₃-C₈)cycloalkyl, pyridyl, benzyl and substituted benzyl wherein the phenyl mojety bears 1 or 2 substituents selected from chloro, bromo, nitro, (C₁-C₄)alkyl and (C₁-C₄)alkoxy.

The term “(C₁-C₄)alkyl” represents straight or branched alkyl chains of from 1 to 4 carbon atoms such as: methyl, ethyl, propyl, 1-methylethyl, butyl, 1-methylpropyl or 1,1-dimethylethyl. The term “(C₃-C₈)cycloalkyl” represents a cycloalkyl group selected from ciclopropyl, ciclobutyl, cyclopentyl, cyclohexyl and cycloheptyl, ciclooctyl. The term “(C₁-C₄)alkoxy” represents a straight or branched alkoxy chain of 1 to 4 carbon atoms such as methoxy, ethoxy, propoxy, 1-methylethoxy, butoxy, 1-methylpropoxy and 1,1-dimethylethoxy.

According to a preferred aspect, the present invention describes novel antibiotic compounds having the general formula (II) wherein one of R₁ or R₂ substituent represents the group —NR₃R₄ whereas the other substituent represents —OH and wherein R₃ and R₄ independently represent:
an alkyl of 1 to 12 carbon atoms;
an alkenyl of 3 to 10 carbon atoms;
a cycloalkyl of 5 to 6 carbon atom optionally substituted by one or two substituents independently selected from lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, lower alkyl of 1 to 4 carbon atoms, and lower alkoxy of 1 to 4 carbon atoms.
a phenyl radical optionally substituted by one or two substituents independently selected from halo, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms.
a benzyl radical optionally substituted on the phenyl ring by one or two substituents independently selected from halo, cyano, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms.
a naphthyl radical optionally substituted by one or two substituents selected from halo, lower alkyl of 1 to 4 carbon atoms, and lower alkoxy of 1 to 4 carbon atoms
a group of formula

—(CH₂)ₙOR₅
in which n represents an integer from 2 to 5 and R₅ represent
   hydrogen or
   (C₁-C₄)alkyl or
   a cycloalkyl of 5 to 6 carbon atom optionally substituted by one or two substituents independently selected from halo, cyano, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms.
   a phenyl radical optionally substituted by one or two substituents independently selected from halo, cyano, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms.
a group of formula

—(CH₂)ₙNR₆R₇
in which n represents an integer from 2 to 8 and R₆ and R₇ independently represent
   hydrogen or
   (C₁-C₄)alkyl or
   a cycloalkyl of 3 to 6 carbon atom optionally substituted by one or two substituents independently selected from halo, cyano, lower alkyl of 1 to 4 carbon atoms, lower alkoxy of 1 to 4 carbon, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon, and lower alkoxy of 1 to 4 carbon atoms.
   a phenyl radical optionally substituted by one or two substituents independently selected from halo, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms.
   a benzyl radical optionally substituted on the phenyl ring by one or two substituents independently selected from halo, cyano, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms.
   R₆ and R₇ taken together represent a —(CH₂)₃, —(CH₂)₄—, —(CH₂)₂—O—(CH₂)₂, —(CH₂)₂—S—(CH₂)₂ or
   R₆ and R₇ taken together with the adjacent nitrogen atom represent: a piperazine mojety which may be substituted in position 4 with a substituent selected from (C₁-C₄)alkyl, (C₃-C₈)cycloalkyl, pyridyl, benzyl and substituted benzyl wherein the phenyl mojety bears 1 or 2 substituents selected from chloro, bromo, nitro, (C₁-C₄)alkyl and (C₁-C₄)alkoxy.

The term “(C₁-C₄)alkyl” represents straight or branched alkyl chains of from 1 to 4 carbon atoms. such as: methyl, ethyl, propyl, 1-methylethyl, butyl, 1-methylpropyl or 1,1-dimethylethyl. The term “(C₃-C₈)cycloalkyl” represents a cycloalkyl group selected from ciclopropyl, ciclobutyl, cyclopentyl, cyclohexyl and cycloheptyl, ciclooctyl. The term “(C₁-C₄)alkoxy” represents a straight or branched alkoxy chain of 1 to 4 carbon atoms such as methoxy, ethoxy, propoxy, 1-methylethoxy, butoxy, 1-methylpropoxy and 1,1-dimethylethoxy.

In another preferred embodiment, the present invention describes novel antibiotic compounds having the general formula (II) wherein one of R₁ or R₂ substituent represents the group —NR₃R₄ whereas the other substituent represents —OH, and wherein R₃ and R₄ independently represent:
a group of formula

—(CH₂)ₙNR₆R₇
in which n represents an integer from 2 to 8 and R₆ and R₇ independently represent
   hydrogen or
   (C₁-C₄)alkyl or
   a cycloalkyl of 3 to 6 carbon atom optionally substituted by one or two substituents independently selected from halo, cyano, lower alkyl of 1 to 4 carbon atoms, lower alkoxy of 1 to 4 carbon, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon, and lower alkoxy of 1 to 4 carbon atoms.
   a phenyl radical optionally substituted by one or two substituents independently selected from halo, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms.
   a benzyl radical optionally substituted on the phenyl ring by one or two substituents independently selected from halo, cyano, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms.
   R₆ and R₇ taken together represent a —(CH₂)₃, —(CH₂)₄—, —(CH₂)₂—O—(CH₂)₂, —(CH₂)₂—S—(CH₂)₂ or
   R₆ and R₇ taken together with the adjacent nitrogen atom represent: a piperazine mojety which may be substituted in position 4 with a substituent selected from (C₁-C₄)alkyl, (C₃-C₈)cycloalkyl, pyridyl, benzyl and substituted benzyl wherein the phenyl mojety bears 1 or 2 substituents selected from chloro, bromo, nitro, (C₁-C₄)alkyl and (C₁-C₄)alkoxy.

The term “(C₁-C₄)alkyl” represents straight or branched alkyl chains of from 1 to 4 carbon atoms. such as: methyl, ethyl, propyl, 1-methylethyl, butyl, 1-methylpropyl or 1,1-dimethylethyl. The term “(C₃-C₈)cycloalkyl” represents a cycloalkyl group selected from ciclopropyl, ciclobutyl, cyclopentyl, cyclohexyl and cycloheptyl, ciclooctyl. The term “(C₁-C₄)alkoxy” represents a straight or branched alkoxy chain of 1 to 4 carbon atoms such as methoxy, ethoxy, propoxy, 1-methylethoxy, butoxy, 1-methylpropoxy and 1,1-dimethylethoxy.

In another preferred embodiment, the present invention describes novel antibiotic compounds having the general formula (II) wherein at least one R₁ and R₂ substituent represents the group —NR₃R₄ whereas the other substituent represents either —OH or —NR₃R₄, and wherein R₃ and R₄ independently represent:
a group of formula

—(CH₂)ₙNR₆R₇
in which n represents an integer from 2 to 4 and R₆ and R₇ independently represent
   hydrogen or (C₁-C₄)alkyl.

The present invention describes also novel antibiotic compounds having the general formula (II) wherein both R₁ and R₂ substituents represent the group —NR₃R₄ and wherein R₃ and R₄ independently represent: hydrogen or
an alkyl of 1 to 20 carbon atoms;
an alkenyl of 2 to 20 carbon atoms;
an alkynyl of 2 to 20 carbon atoms;
a cycloalkyl of 3 to 8 carbon atom optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms;
a phenyl radical optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms
a benzyl radical optionally substituted on the phenyl ring by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms
a naphthyl radical optionally substituted by one or two substituents selected from halo, lower alkyl of 1 to 4 carbon atoms optionally substituted by 1 to 3 halogen atoms, and lower alkoxy of 1 to 4 carbon atoms optionally substituted by 1 to 3 halogen atoms
a group of formula

—(CH₂)ₙOR₅
in which n represents an integer from 2 to 8 and R₅ represent
   hydrogen or
   (C₁-C₄)alkyl or
   a cycloalkyl of 3 to 8 carbon atom optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms.
   a phenyl radical optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms
a group of formula

—(CH₂)ₙNR₆R₇
in which n represents an integer from 2 to 8 and R₆ and R₇ independently represent
   hydrogen or
   (C₁-C₄)alkyl or
   a cycloalkyl of 3 to 8 carbon atom optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms.
   phenyl radical optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms
   a benzyl radical optionally substituted on the phenyl ring by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms
   R₆ and R₇ taken together represent a —(CH₂)₃, —(CH₂)₄—, —(CH₂)₂—O—(CH₂)₂, —(CH₂)₂—S—(CH₂)₂ or
   R₆ and R₇ taken together with the adjacent nitrogen atom represent: a piperazine mojety which may be substituted in position 4 with a substituent selected from (C₁-C₄)alkyl, (C₃-C₈)cycloalkyl, pyridyl, benzyl and substituted benzyl wherein the phenyl mojety bears 1 or 2 substituents selected from chloro, bromo, nitro, (C₁-C₄)alkyl and (C₁-C₄)alkoxy.

The term “(C₁-C₄)alkyl” represents straight or branched alkyl chains of from 1 to 4 carbon atoms. such as: methyl, ethyl, propyl, 1-methylethyl, butyl, 1-methylpropyl or 1,1-dimethylethyl. The term “(C₃-C₈)cycloalkyl” represents a cycloalkyl group selected from ciclopropyl, ciclobutyl, cyclopentyl, cyclohexyl and cycloheptyl, ciclooctyl. The term “(C₁-C₄)alkoxy” represents a straight or branched alkoxy chain of 1 to 4 carbon atoms such as methoxy, ethoxy, propoxy, 1-methylethoxy, butoxy, 1-methylpropoxy and 1,1-dimethylethoxy.

In another preferred embodiment, the present invention describes novel antibiotic compounds having the general formula (II) wherein both R₁ and R₂ substituents represent the group —NR₃R₄ and wherein R₃ and R₄ independently represent:
an alkyl of 1 to 12 carbon atoms;
an alkenyl of 3 to 10 carbon atoms;
a cycloalkyl of 5 to 6 carbon atom optionally substituted by one or two substituents independently selected from lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, lower alkyl of 1 to 4 carbon atoms, and lower alkoxy of 1 to 4 carbon atoms.
a phenyl radical optionally substituted by one or two substituents independently selected from halo, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms.
a benzyl radical optionally substituted on the phenyl ring by one or two substituents independently selected from halo, cyano, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms.
a naphthyl radical optionally substituted by one or two substituents selected from halo, lower alkyl of 1 to 4 carbon atoms, and lower alkoxy of 1 to 4 carbon atoms
a group of formula

—(CH₂)ₙOR₅
in which n represents an integer from 2 to 5 and R₅ represent
   hydrogen or
   (C₁-C₄)alkyl or
   a cycloalkyl of 5 to 6 carbon atom optionally substituted by one or two substituents independently selected from halo, cyano, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms.
   a phenyl radical optionally substituted by one or two substituents independently selected from halo, cyano, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms.
a group of formula

—(CH₂)ₙNR₆R₇
in which n represents an integer from 2 to 8 and R₆ and R₇ independently represent
   hydrogen or
   (C₁-C₄)alkyl or
   a cycloalkyl of 3 to 6 carbon atom optionally substituted by one or two substituents independently selected from halo, cyano, lower alkyl of 1 to 4 carbon atoms, lower alkoxy of 1 to 4 carbon, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon, and lower alkoxy of 1 to 4 carbon atoms.
   a phenyl radical optionally substituted by one or two substituents independently selected from halo, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms.
   a benzyl radical optionally substituted on the phenyl ring by one or two substituents independently selected from halo, cyano, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms.
   R₆ and R₇ taken together represent a —(CH₂)₃, —(CH₂)₄—, —(CH₂)₂—O—(CH₂)₂, —(CH₂)₂—S—(CH₂)₂ or
   R₆ and R₇ taken together with the adjacent nitrogen atom represent: a piperazine mojety which may be substituted in position 4 with a substituent selected from (C₁-C₄)alkyl, (C₃-C₈)cycloalkyl, pyridyl, benzyl and substituted benzyl wherein the phenyl mojety bears 1 or 2 substituents selected from chloro, bromo, nitro, (C₁-C₄)alkyl and (C₁-C₄)alkoxy.

The term “(C₁-C₄)alkyl” represents straight or branched alkyl chains of from 1 to 4 carbon atoms. such as: methyl, ethyl, propyl, 1-methylethyl, butyl, 1-methylpropyl or 1,1-dimethylethyl. The term “(C₃-C₈)cycloalkyl” represents a cycloalkyl group selected from ciclopropyl, ciclobutyl, cyclopentyl, cyclohexyl and cycloheptyl, ciclooctyl. The term “(C₁-C₄)alkoxy” represents a straight or branched alkoxy chain of 1 to 4 carbon atoms such as methoxy, ethoxy, propoxy, 1-methylethoxy, butoxy, 1-methylpropoxy and 1,1-dimethylethoxy.

In yet another preferred embodiment, the present invention describes novel antibiotic compounds having the general formula (II) wherein both R₁ and R₂ substituents represent the group —NR₃R₄ and wherein R₃ and R₄ independently represent:
a group of formula

—(CH₂)ₙNR₆R₇
in which n represents an integer from 2 to 8 and R₆ and R₇ independently represent
   hydrogen or
   (C₁-C₄)alkyl or
   a cycloalkyl of 3 to 6 carbon atom optionally substituted by one or two substituents independently selected from halo, cyano, lower alkyl of 1 to 4 carbon atoms, lower alkoxy of 1 to 4 carbon, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon, and lower alkoxy of 1 to 4 carbon atoms.
   a phenyl radical optionally substituted by one or two substituents independently selected from halo, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms.
   a benzyl radical optionally substituted on the phenyl ring by one or two substituents independently selected from halo, cyano, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms.
   R₆ and R₇ taken together represent a —(CH₂)₃, —(CH₂)₄—, —(CH₂)₂—O—(CH₂)₂, —(CH₂)₂—S—(CH₂)₂ or
   R₆ and R₇ taken together with the adjacent nitrogen atom represent: a piperazine moiety which may be substituted in position 4 with a substituent selected from (C₁-C₄)alkyl, (C₃-C₈)cycloalkyl, pyridyl, benzyl and substituted benzyl wherein the phenyl mojety bears 1 or 2 substituents selected from chloro, bromo, nitro, (C₁-C₄)alkyl and (C₁-C₄)alkoxy.

The term “(C₁-C₄)alkyl” represents straight or branched alkyl chains of from 1 to 4 carbon atoms. such as: methyl, ethyl, propyl, 1-methylethyl, butyl, 1-methylpropyl or 1,1-dimethylethyl. The term “(C₃-C₈)cycloalkyl” represents a cycloalkyl group selected from ciclopropyl, ciclobutyl, cyclopentyl, cyclohexyl and cycloheptyl, ciclooctyl. The term “(C₁-C₄)alkoxy” represents a straight or branched alkoxy chain of 1 to 4 carbon atoms such as methoxy, ethoxy, propoxy, 1-methylethoxy, butoxy, 1-methylpropoxy and 1,1-dimethylethoxy.

In yet another preferred embodiment, the present invention describes novel antibiotic compounds having the general formula (II) wherein both R₁ and R₂ substituents represent the group —NR₃R₄ and wherein R₃ and R₄ independently represent:
a group of formula

—(CH₂)ₙNR₆R₇
in which n represents an integer from 2 to 4 and R₆ and R₇ independently represent
   hydrogen or (C₁-C₄)alkyl.

It has to be noted that, when both R₁ and R₂ substituents represent the group —OH, general formula (II) provides the revised structure of the lantibiotic 97518, which has two carboxylic functions (the 14-Glu side chain and 24-Ala carboxy terminal) and a primary amino function (N-terminal 1-Ile). emerge

Experimental evidence of the revised structure of 97518 emerged by MS/MS experiments on 97518 (see Table 3) and was confirmed by NMR analysis of the diamide derivative 13 (see Table 5). These data allowed a new interpretation of the previously reported NMR assignments (Biochemistry 2007, 46, 5884-5895). The new NMR assignments are reported in Table 2.

97518 in its revised structure, is thus represented by formula III.

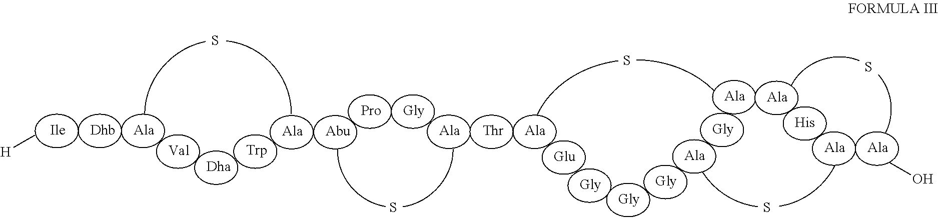

Thus compounds according to the invention are mono or diamide derivatives of 97518.

More particularly, they are mono or di-basic amide derivatives of 97518, which may be schematically represented by formula (II). The novel compounds of formula (II) of this invention generally exhibit an improved antimicrobial activity in respect of 97518.

Among the novel compounds of formula (II) according to the present invention, are preferred those compounds wherein at least one of the amides moieties —NR₃R₄ has the following formula:

—NH—(CH₂)₂—NH₂; —NH(CH₂)₃NH₂—NH—(CH₂)₄—NH₂; —NH(CH₂)₃NHCH₃—NH—(CH₂)₃—N(CH₃)₂; —NH—(CH₂)₃N(C₂H₅)₂—NH—(CH₂)₃N(C₃H₇)₂; —NH—(CH₂)₃N(C₄H₉)₂—NH—(CH₂)₅N(CH₃)₂; —NH(CH₂)₆N(CH₃)₂—NH(CH₂)₆NHCH₃; —N[(CH₂)₂NH₂]₂—N[(CH₂)₃NH₂]₂; —N[(CH₂)₂N(CH₃)₂]₂N[(CH₂)₃N(CH₃)₂]₂; —N[(CH₂)₄NH₂]₂

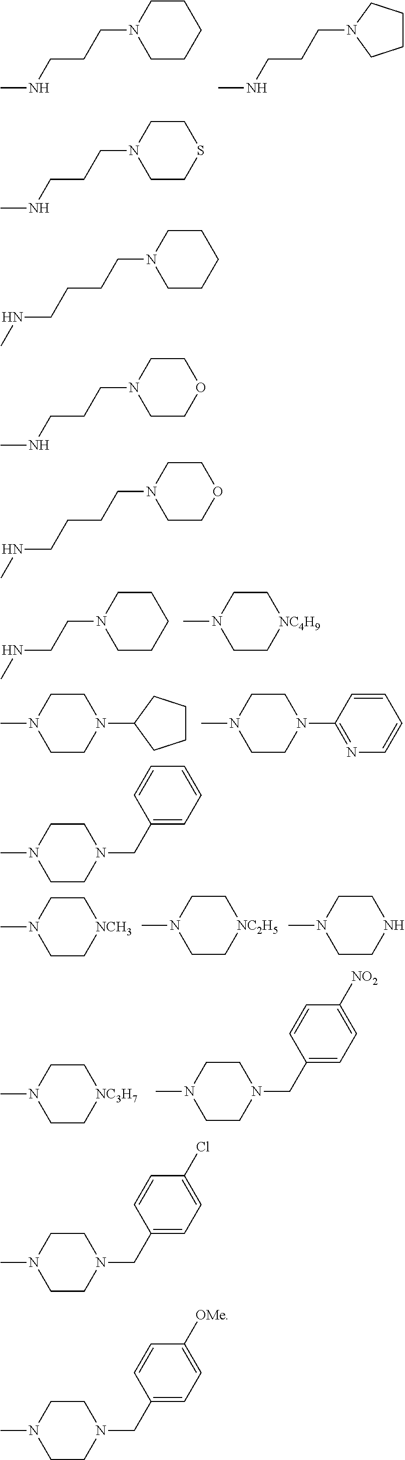

The compounds of the present invention possess ionizable functions and are thus capable of forming salts. Preferred addition salts of the compounds of the present invention are the “pharmaceutically acceptable acid addition salts” which are intended as those salts with acids which from a biological, manufacturing and formulation standpoint are compatible with the pharmaceutical practice as well as with the use in animals. Representative and suitable acid addition salts of the compounds of formula (II) include those salts formed by standard reaction with both organic and inorganic acids such as, for example, hydrochloric, hydrobromic, sulfuric, phosphoric, acetic, trifluoroacetic, trichloroacetic, succinic, citric, ascorbic, lactic, maleic, fumaric, palmitic, cholic, pamoic, mucic, glutamic, camphoric, glutaric, glycolic, phtalic, tartaric, lauric, stearic, salicylic, methanesulfonic, dodecylsulfonic (estolic), benzenesulfonic, sorbic, picric, benzoic, cinnamic acid and the like. The transformation of the free amino or nonsalts compounds of the present invention into the corresponding addition salts, and the reverse, i.e. the transformation of an addition salts of a compound of the invention into the non-salt or free amino form, are within the ordinary technical skill and are encompassed by the present invention. For instance, a free amino compound of formula (II) can be transformed into the corresponding acid addition-salt by dissolving the non-salt form in an aqueous solvent and adding a slight molar excess of the selected acid. The resulting solution or suspension is then lyophilized to recover the desired salt. Instead of lyophilizing, in some instances, it is possible to recover the final salt by extraction, from an aqueous solution thereof with a water immiscible organic solvent wherein the salt form is soluble, concentration to a small volume of the separated organic phase and precipitation by adding a non-solvent. In case the final salt is insoluble in an organic solvent where the non-salt form is soluble, it may be recovered by filtration from the organic solution of the non-salt form after addition of the stoichiometric amount or a slight excess of the selected acid.

The non-salt form can be prepared from a corresponding acid salt dissolved in an aqueous solvent, which is then neutralized to free the nonsalt form. This latter is recovered, for instance, by extraction with a water immiscible organic solvent or is transformed into another acid addition salt by adding the selected acid and working up as above. A common desalting procedure may be employed when, following the neutralization, desalting is necessary.

For example, column chromatography on controlled pore polydextrane resins (such as Sephadex LH 20) or silanized silica gel may be conveniently used. After eluting the undesired salts with an aqueous solution, the desired product is eluted by means of linear or step gradient of a mixture of water and a polar or apolar organic solvent. As known in the art, the salt formation with either pharmaceutically or nonpharmaceutically acceptable acids may be used as a convenient purification technique. After formation and isolation, the salt form of a compound of formula (II) can be transformed into the corresponding non-salt or into a pharmaceutically acceptable salt. In some instances the acid addition salt of a compound of formula (II) is more soluble in water and hydrophilic solvents and has an increased chemical stability. Good solubility and stability in water or hydrophilic solvents of an active compound are in general appreciated in the art, for the preparation of suitable pharmaceutical compositions for the administration of the medicament. However, in view of the similarity of the properties of the compounds of formula (II) with their salts, what is said in the present application when dealing with the biological activities of the non-salt compounds of formula (II) applies also to their pharmaceutically acceptable salts, and vice versa.

The compounds of the present invention can be administered orally, topically or parenterally, the preferred route of administration depending on the treatment to be carried out. Depending on the route of administration, these compounds can be formulated into various dosage forms. Preparations for oral administration may be in the form of capsules, tablets, liquid solutions or suspensions. As known in the art, the capsules and tablets may contain in addition to the active ingredient conventional excipients such as diluents e.g. lactose, calcium phosphate, sorbitol and the like lubricants e.g. magnesium stearate, talc, polyethylene glycol, binding agents, e.g. polyvinylpyrrolidone, gelatin, sorbitol, tragacanth, acacia, flavoring agents, and acceptable disintegrating and wetting agents. The liquid preparations generally in the form of aqueous or oily solutions or suspensions may contain conventional additives such as suspending agents. For topical use the compounds of formula (II) of the present invention may also be prepared in suitable forms for absorption through the mucous membranes of the nose and throat or bronchial tissues and may conveniently take the form of liquid sprays or inhalants lozenges or throat paints. For medication of the eyes, the preparation may be presented in liquid or semi-liquid form. Topical applications may be formulated in hydrophobic or hydrophilic bases as ointments, creams, lotions, paints, or powders. For rectal administration the compounds of formula (II) of the invention are administered in the form of suppositories admixed with conventional vehicles, such as, for example, cocoa butter, wax, spermaceti or polyethylenglycols and their derivatives. Compositions for injection may take such forms as suspensions, solutions, or emulsions in oily or aqueous vehicles, and may contain formulatory agents such as suspending, stabilizing and/or dispersing agents. Alternatively, the active ingredient may be in powder form for reconstitution at the time of delivery with a suitable vehicle, such as sterile water. The amount of active principle to be administered depends on various factors such as the size and conditions of the subject to be treated, the route and frequency of administration, and the causative agent involved.

The compounds of the invention are generally effective at a dosage comprised between about 1 and 30 about 40 mg of active ingredient per Kg of body weight. Depending on the characteristics of the specific compound, the infection and the patients, the effective dose can be administered in a single administration per day or divided in 2 to 4 administrations per day. Particularly desirable compositions are those prepared in the form of dosage units containing from about 30 to about 500 mg per unit.

The compounds of the present invention can also be employed in combination with other approved drugs, being that another antibacterial agent or an agent intended to treat a second symptom or the cause of a different condition. For example, the antibacterial agents that can be used in conjunction with the compounds of the present invention include but are not limited to quinolones, tetracyclines, glycopeptides, aminoglycosides, β-lactams, rifamycins, coumermycins, macrolides, ketolides, azalides, oxazolidinones, lipopeptides and chloramphenicol. Therefore, compositions of the compounds of the present invention with other approved drugs fall also within the scope of the present invention.

The novel compounds of formula (II) of the present invention, including salts, formulation and compositions thereof, can be effectively employed as the active ingredients of the antimicrobial preparations used in human or animal medicine for the prevention and treatment of infectious diseases caused by pathogenic bacteria which are susceptible to said active ingredients, in particular, for the treatment of infections caused by enterococci, streptococci and staphylococci.

The invention also provides the use of a compound or composition thereof for the manufacture of a medicament for use in a specific method of treatment or prophylaxis of the human or animal body, the specific method including those described herein below.

Thus the compounds or compositions thereof of the invention may be used for the treatment of bacterial infections, including systemic bacterial infections, caused by bacteria including Clostridium difficile, Staphylococcus spp., Streptococcus spp, Enterococcus spp, Propionibacterium acnes, and Moraxella spp.

The variants and composition may be used for systemic treatment of bacteraemia (including catheter related bacteraemia), pneumonia, skin and skin structure infections (including surgical site infections), endocarditis and osteomyelitis. The variants or compositions may also be used for topical treatment of skin infections including impetigo and acne. The variants and compositions thereof may also be used in the treatment of eye infections, such as conjunctivitis, and for oral treatment for gut super-infection, such as that caused by Clostridium difficile.

The compounds may also be used in the treatment or prevention of infection of the skin in wounds or burns. In addition, the variants and compositions thereof may be used in prophylactic methods, such as for the clearance of the nostrils to prevent transmission of MRSA. This may be practiced on subjects at risk of infection (e.g. patients entering a hospital) or on health professionals or others at risk of being carriers of such infections. Prophylactic clearance of gut flora ahead of abdominal surgery is also contemplated.

The invention also relates the preparation of the novel compounds of formula II The amidation procedure involves condensing said 97518, for example of formula (III), with a selected amine of general formula HNR₃R₄, wherein R₃ and R₄ are as defined above, in the presence of a condensing agent in the presence of a solvent.

Inert organic aprotic solvents useful for the condensation reaction are those solvents which do not unfavorably interfere with the reaction course and are capable of at least partially solubilizing the starting material, for example the antibiotic of formula (III). Examples of said solvents are organic amides, ethers of glycols and polyols, phosphoramide derivatives, sulfoxides. Preferred solvents are: dimethylformamide, dimethoxyethane, hexamethyl phosphoroamide, dimethylsulphoxide, dioxane, N-methylpyrrolidone and mixtures thereof. Preferably, dimethylformamide (DMF) is employed.

The condensing agent according to the present invention, is one suitable for forming amide bonds in organic compounds and, in particular, in peptide synthesis. Representative examples of condensing agents are diisopropylcarbodiimide (DIC), dicyclohexylcarbodiimide (DCC) without or in the presence of hydroxybenzotriazole (HOBT), N,N,N′,N′-tetramethyl-O-(benzotriazol-1-yl)uronium tetrafluoroborate (TBTU), N,N,N′,N′-tetramethyl-O-(7oxabenzotriazol-1-yl)uronium hexafluorophosphate (HATU), benzotriazolyloxy-tris-(dimethylamino)phosphonium hexafluorophosphate (HBTU), benzotriazolyloxy-tris-(pyrrolidino)pho-sphonium hexafluorophosphate (PyBOP) and (C₁-C₄)alkyl, phenyl or heterocyclic phosphorazidates such as diphenylphosphorazidate, dimorpholyl-phosphorazidate. The preferred condensing agent is PyBOP. The condensing agent is generally employed in a slight molar excess, such as from 2.2 to 5; preferably the molar excess of condensing agent is about 2.5 times the molar amount of antibiotic starting compound of formula (III). According to the present method, the amine is normally used in slight molar excess with respect to the compound of formula (III). In general, a 2 to 40-fold molar excess of the selected amine is used, while a 15-30 fold molar excess is preferred.

When the amine R₃R₄NH is reacted as a corresponding salt, for example the hydrochloride salt, it is necessary to add a suitable base in at least a molar proportion to obtain the free base of the amine R₃R₄NH which reacts with 97518. In this case, an excess of the base is generally preferred. It is convenient to add a salt-forming base to the reaction mixture in an at least equimolecular amount, and preferably in about 1.2 fold molar excess with respect to the amine R₃R₄NH. Examples of said salt-forming bases are tertiary organic aliphatic or alicyclic amines such as trimethylamine, triethylamine (TEA), N-methylpyrrolidine or heterocyclic bases such as picoline and the like, alkali metals (e.g. sodium and potassium) hydrogen carbonates and carbonates.

The reaction temperature will vary considerably depending on the specific starting materials and reaction conditions. In general, it is preferred to conduct the amidation reaction at temperature from 0° C. to 50° C. preferably at room temperature.

Also the reaction time varies considerably, depending on the other reaction parameters; in general the condensation is completed in about 2-4 h.

When the amine R₃R₄NH contains a further primary amino group it might be protected, if necessary, as known in the art, in order to get the desired product. Any typical protecting group of the amino rest, which is resistant to the conditions applied during the process of this invention and may be readily removed under conditions which do not affect the stability of the 97518 core portion can be utilized here. Suitable protecting groups of the amino function can be selected, for instance, from the groups described in: T. W. Greene, “Protective Groups in Organic Synthesis”, J. Wiley, N.Y., 1981. In particular, in this case, those protecting groups, which are formed by acylating the amino moiety, are preferred. The protecting groups employed in the process herein described are those generally employed in peptides synthesis. Obviously, a deprotection step is then necessary to obtain the desired final product.

Generally, the reaction course is monitored by HPLC according to methods known in the art. On the basis of the results of this assays it will be possible to evaluate the reaction course and decide when to stop the reaction and start working up the reaction mass according to per se known techniques which include, for instance, precipitation by addition of non-solvents, extraction with solvents, in conjunction with further common separation operations and purification, e.g. by column chromatography.

Mono amidation can be obtained by using a sub-stoichiometric amount of amine NHR₃R₄. The amine is normally used in 0.5-1 fold molar amount with respect to the compound of formula (III). The two monoamide derivatives can be purified according to per se known techniques which include, for instance column chromatography.

According to the methodologies of the present invention as well as according to the above Examples, a series of compounds can be prepared, as summarized in Table 1.

TABLE 1

| | R₁ | R₂ |
|---|---|---|
| 97518 | OH | OH |
| 1 | 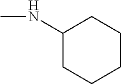 | 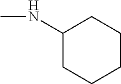 |
| 2 | 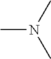 | 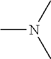 |
| 3 | 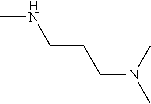 | 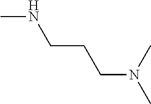 |
| 4 | 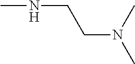 | 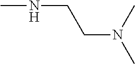 |
| 5 | 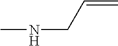 | 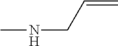 |
| 6 | 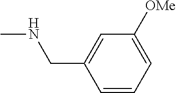 | 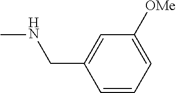 |
| 7 | 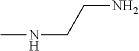 | 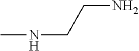 |
| 8 | 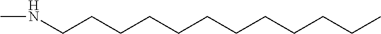 | 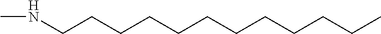 |
| 9 | 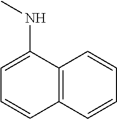 | 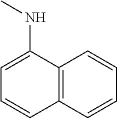 |
| 10 | 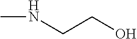 | 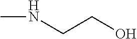 |
| 11 | 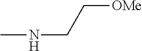 | 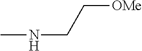 |
| 12 | 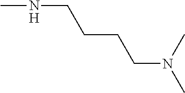 | 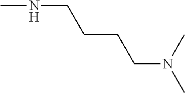 |
| 13 | 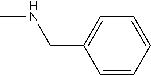 | 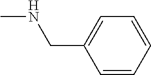 |
| 14 | 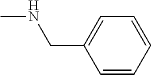 | OH |
| 15 | OH | 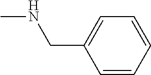 |
| 16 | 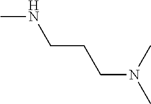 | OH |
| 17 | OH | 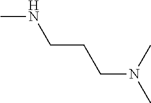 |
| 18 | 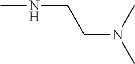 | OH |
| 19 | OH | 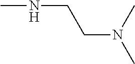 |
| 20 | 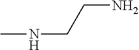 | OH |
| 21 | OH | 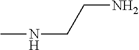 |
| 22 | 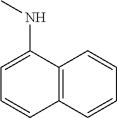 | OH |
| 23 | OH | 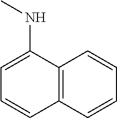 |
| 24 | 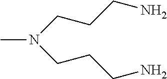 | 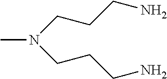 |
| 25 | 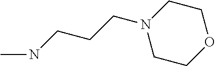 | 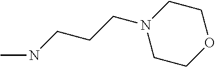 |
| 26 | 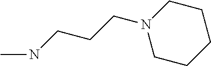 | 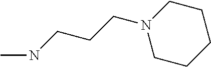 |
| 27 | 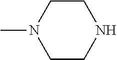 | 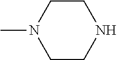 |
| 28 | 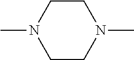 | 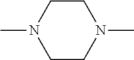 |
| 29 | 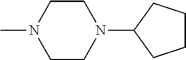 | 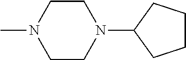 |
| 30 | 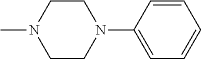 | 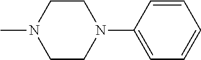 |
| 31 | 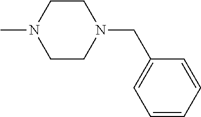 | 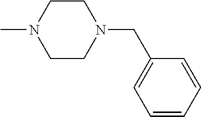 |

### EXAMPLES

### Example 1

### Structure of Lantibiotic 97518

NMR Spectroscopy. NMR spectroscopic analyses were performed on samples of 6.1 mg of 97518 in 0.5 mL H2O/D2O 9:1 (v/v) added with 1.5 μL of DCl and supplemented with 20 μL of acetonitrile to solubilize the antibiotic. The H 1D spectrum (using water suppression by Excitation Sculpting), two-dimensional DQF-COSY, TOCSY, and NOESY experiments were performed at 283, 298 and 313 K using a Bruker Avance 600 MHz spectrometer. For the TOCSY experiments we used a mixing time of 20, 60 and 100 ms was used whereas NOESY spectra were acquired with 300 and 700 ms mixing times. Natural abundance heteronuclear ¹³C-¹H HSQC (J=145), HMBC (J^{1H-13C}=8 Hz), ¹H-¹⁵N HSQC (J=90 Hz) and ¹H-¹⁵N HSQC-TOCSY experiments were performed.

The complete assignment of 97518 is reported in table 2.

TABLE 2
complete NMR-signals assignment for 97518

| residue | NH(¹⁵N) | H_{α} | H_{β} | H_{γ} | others |
|---|---|---|---|---|---|
| 1-Ile | — | 4.18 | 2.19 | 1.61-1.17 | 1.01 |
| 2-Dhb | 10.00(124.1) | | 6.68 | 1.88 | |
| 3-Ala | 8.32(115.5) | 4.68 | 3.38-3.15 | | |
| 4-Val | 8.13(118.3) | 4.18 | 2.3 | 0.98 | |
| 5-Dha | 9.14(126.2) | 5.92-5.42 | | | |
| 6-Trp | 8.7(120.3) | 4.67 | 3.48-3.39 | | 7.76-7.34-7.23 |
| 7-Ala | 8.29(120.2) | 4.67 | 3.15 | | |
| 8-Abu | 8.93(115.3) | 5.1 | 3.63 | 1.37 | |
| 9-Pro | — | 4.43 | 2.40 | 1.91-1.85 | 3.53-3.34 |
| 10-Gly | 9.23(113.3) | 4.1-3.95 | | | |
| 11-Ala | 8.32(115.6) | 4.08 | 3.12 | | |
| 12-Thr | 8.43(114.3) | 4.37 | 4.29 | 1.28 | |
| 13-Ala | 8.69(121.4) | 4.66 | 3.15-3.05 | | |
| 14-Glu | 8.41(120.8) | 4.32 | 2.16-2.10 | 2.51 | |
| 15-Gly | 8.80(111.4) | 4.1-3.91 | | | |
| 16-Gly | 8.01(106.8) | 4.05-3.8 | | | |
| 17-Gly | 8.32(108.1) | 4.1-3.9 | | | |
| 18-Ala | 8.96(121.2) | 4.44 | 3.19 | | |
| 19-Gly | 8.82(108.7) | 4.37-3.8 | | | |
| 20-Ala | 8.26(117.3) | 4.43 | 3.39-2.97 | | |
| 21-Ala | 8.19(113.3) | 5.17 | 3.21-3.14 | | |
| 22-His | 8.99(123.2) | 4.57 | 3.52 | | 8.74-7.45 |
| 23-Ala | 9.13(115.1) | 4.69 | 3.48-2.67 | | |
| 24-Ala | 7.52(123.0) | 4.14 | 3.77-2.99 | | |

Mass spectrometry: The MS spectra were obtained by electrospray ionization in positive mode by direct infusion using a Bruker Esquire 3000 plus, with ion trap. The double charged ion corresponding to 97518 show a MS peak as double charged ion with 1097.7 m/z. MS/MS analysis of the double charged ion was performed at 0.7, 1.2 and 2 V. In table 3 all the observed fragmentations were reported with their assignment.

TABLE 3
MS/MS ESI⁺ of 1097.7 m/z

| m/z | Fragment/ion |
|---|---|
| 2076.9 | 1-23(-Ala(OH)SH) |
| 1828.9 | 5-24(SH) |
| 1573.7 | 7-24(SH) |
| 1470.7 | 8-24 |
| 1387.6 | 9-24(SH) |
| 1130.7 | 12-24 |
| 1097.7 | [M + 2H]²⁺ |
| 1088.6 | [M-18 + 2H]²⁺ |
| 1039.1 | [M-Ala(OH)SH + 2H]²⁺ |
| 694.5 | [9-24(SH)-18 + 2H]²⁺ |

### Example 2

### Synthesis of Compounds 1 and 6

To a stirred solution of 14.3 mg of 97518 (6.5 μmol) in 350 μl of DMF, 15 μL of cyclohexylamine or 3-methoxy-benzylamine (for the synthesis of compounds 1 and 6, respectively) and 9 mg of PyBOP (17 μmol) were added and the reaction mixture was kept under stirring at room temperature after which HPLC monitor showed completeness (see Table 4). The reaction was quenched by addition of 2N HCl (100 μL) until neutral pH and then diluted with water to 450 μL. The filtered solid redissolved in a mixture of MeCN/H₂O TFA 0.1%=1/1 and lyophilized. The final compounds were analysed by Liquid Chromatography—Mass Spectrometry (Table 4)

### Example 3

### Synthesis of Compound 2

To a stirred solution of 14.3 mg of 97518 (6.5 μmol) in 350 μl of DMF, 30 μL of a 33% EtOH solution of Me₂NH and 9 mg of PyBOP (17 μmol) were added. The reaction mixture was kept under stirring at room temperature after which HPLC monitor showed completeness (see Table 4). The reaction was quenched by addition of HCl 2N (100 μL) until neutral pH and then diluted with water to 450 μL. The filtered solid redissolved in a mixture of MeCN/H₂O TFA 0.1%=1/1 and lyophilized. The final product has been analysed by LC-MS (Table 4).

### Example 4

### Synthesis of Compounds 3, 4, 5, 7, 10, 11, 12

To a stirred solution of 14.3 mg of 97518 (6.5 μmol) in 350 μl of DMF, 10 μL of the suitable amine and 9 mg of PyBOP (17 μmol) were added. The reaction mixture was kept under stirring at room temperature after which HPLC monitor showed completeness (see Table 4). The reaction was quenched by addition of HCl 2N (100 μL) until neutral pH and then diluted with water to 450 μL. The filtered solid redissolved in a mixture of MeCN/H₂O TFA 0.1%=1/1 and lyophilized. The final product has been analysis by MS (Table 4).

### Example 5

### Synthesis of Compounds 8, 9

To a stirred solution of 14.3 mg of 97518 (6.5 μmol) and 15 mg of dodecylamine or 1-naphtylamine (the synthesis of compounds 8 and 9, respectively) in 350 μl of DMF 9 mg of PyBOP (17 μmol) were added and the reaction mixture was kept under stirring at room temperature after which HPLC monitor showed completeness (see Table 4). The reaction was quenched by addition of HCl 2N (100 μL) until neutral pH and then diluted with water to 450 μL. The filtered solid redissolved in a mixture of MeCN/H₂O TFA 0.1%=1/1 and lyophilized. The final product has been analysis by MS (Table 4).

### Example 6

### Synthesis of Compound 13

To a stirred solution of 30 mg of 97518 (13 μmol) and 20 μL of BnNH2 in 300 μl of DMF 14.2 mg of PyBOP (27 μmol) were added and the reaction mixture was kept 1 h under stirring at room temperature. HPLC monitor showed completeness after 2 h (see Table 4). The reaction was diluted with water and the pH was corrected to 3-4 by adding 20 μL of formic acid. The filtered solid was dissolved in 1 mL of a mixture of MeCN/H₂O TFA 0.1%=1/1 and lyophilized. The final product has been analysis by MS (Table 4) and by NMR (Table 5)

TABLE 4
HPLC and MS data of compounds 1-13.

| | HPLC-RT^{(a)} | HPLC-RT^{(b)} | Molecular | Exact | Detected MS ion |
|---|---|---|---|---|---|
| Compound | (minutes) | (minutes) | formula | mass | [M + 2H]²⁺ |
| 1 | 20.5 | 4.6 | C₁₀₂H₁₄₇N₂₉O₂₆S₅ | 2353.96 | 1178 |
| 2 | 16.5 | 4.1 | C₉₄H₁₃₅N₂₉O₂₆S₅ | 2245.87 | 1124 |
| 3 | 15.5 | 3.8 | C₁₀₀H₁₄₉N₃₁O₂₆S₅ | 2359.98 | 1181 |
| 4 | 16 | 3.8 | C₉₈H₁₄₅N₃₁O₂₆S₅ | 2331.95 | 1167 |
| 5 | 17.2 | 4.2 | C₉₆H₁₃₅N₂₉O₂₆S₅ | 2269.87 | 1136 |
| 6 | 20.5 | 4.5 | C₁₀₆H₁₄₃N₂₉O₂₈S₅ | 2429.92 | 1216 |
| 7 | 15.5 | 3.2 | C₉₄H₁₃₇N₃₁O₂₆S₅ | 2275.89 | 1139 |
| 8 | 18 | 5.7 | C₁₁₄H₁₇₅N₂₉O₂₆S₅ | 2526.18 | 1264 |
| 9 | 18.5 | 4.4 | C₁₁₀H₁₃₉N₂₉O₂₆S₅ | 2441.90 | 1222 |
| 10 | 15.6 | 3.4 | C₉₄H₁₃₅N₂₉O₂₈S₅ | 2277.86 | 1140 |
| 11 | 16.7 | 3.5 | C₉₆H₁₃₉N₂₉O₂₈S₅ | 2305.89 | 1154 |
| 12 | 15.7 | 3.2 | C₁₀₂H₁₅₃N₃₁O₂₆S₅ | 2388.02 | 1195 |
| 13 | 19.5 | 4.3 | C₁₀₄H₁₃₉N₂₉O₂₆S₅ | 2369.90 | 1186 |

^{(a)}Instrument: Shimadzu 2010; Column: Merck Lichrosphere RP-18, 4 × 125 mm, 5 μm; flow: 1 mL/min; sample concentration 1 mg/mL; detection λ = 230; inj.vol. = 20 μL; phase A: Trifluoroacetic 0.1%; phase B: acetonitrile. Linear gradient: Time = 0 minutes B = 10%; T = 40 minutes B = 90%; followed by 10 minutes for reequilibration with B = 10%
^{(b)}Instrument: Agilent 1100 and Bruker Esquire 3000+ ion trap (ESI+ interface); Column: Waters Atlantis 50 × 4.6 mm, 3 um flow: 1 mL/min, 0.5 mL/min to MS; phase A: Water (MilliQ) + 0.05% TFA; phase B: Acetonitrile + 0.05% TFA; gradient: 10-90% B in 6 min, 1 min washing at 100% B, 3 min reequilibration at 10% B.

TABLE 5
complete NMR-signals assignment for compound 13

| residue | NH | H_{α} | H_{β} | H_{γ} | others |
|---|---|---|---|---|---|
| 1-Ile | — | 4.18 | 2.19 | 1.61-1.17 | 1.01 |
| 2-Dhb | 9.99 | | 6.66 | 1.86 | |
| 3-Ala | 8.03 | 4.68 | 3.31-3.08 | | |
| 4-Val | 8.13 | 4.15 | 2.29 | 0.98 | |
| 5-Dha | 8.76 | 6.11-5.42 | | | |
| 6-Trp | 8.57 | 4.56 | 3.46-3.39 | | 7.76-7.34- |
| | | | | | 7.23 |
| 7-Ala | 8.26 | 4.66 | 3.14 | | |
| 8-Abu | 8.94 | 5.1 | 3.63 | 1.37 | |
| 9-Pro | — | 4.43 | 2.40 | 1.91-1.85 | 3.53-3.34 |
| 10-Gly | 9.15 | 4.1-3.95 | | | |
| 11-Ala | 8.32 | 4.08 | 3.12 | | |
| 12-Thr | 8.43 | 4.37 | 4.29 | 1.28 | |
| 13-Ala | 8.58 | 4.66 | 3.14-3.06 | | |
| 14-Glu | 8.31 | 4.26 | 2.16-2.10 | 2.42 | |
| NH—CH₂—Ph | 8.12 | 4.43 | | | 7.4-7.3 |
| 15-Gly | 8.65 | 4.1-3.91 | | | |
| 16-Gly | 7.94 | 4.00-3.73 | | | |
| 17-Gly | 8.19 | 4.04-3.84 | | | |
| 18-Ala | 8.85 | 4.44 | 3.18 | | |
| 19-Gly | 8.69 | 4.31-3.77 | | | |
| 20-Ala | 8.20 | 4.38 | 3.35-2.97 | | |
| 21-Ala | 8.13 | 5.14 | 3.22-3.14 | | |
| 22-His | 8.96 | 4.57 | 3.52 | | 8.74-7.45 |
| 23-Ala | 9.11 | 4.69 | 3.48-2.62 | | |
| 24-Ala | 7.46 | 3.97 | 3.65-2.95 | | |
| NH—CH₂—Ph | 8.53⁽*⁾ | 4.41 | | | 7.47-7.37- |
| | | | | | 7.33 |

⁽*⁾The pattern of NOE signals correlating with this NH is composed by three peaks (one H_{α} and two H_{β}) thus proving the 24-Ala carboxy terminal amminoacid of the revised structure while it is not compatible with the spin system of a 24-Gly carboxy terminal amminoacid as previously reported.

### Example 7

### Antibacterial Activity

The antimicrobial activity of the compounds prepared as described in Examples 2-6 was evaluated against a panel of clinical isolates of methicillin-sensitive, methicillin-resistant, vancomycin-intermediate S. aureus, Van-S and Van-A Enterococcus faecium and faecalis, Streptococcus pyogenes, Escherichia coli and Candida albicans. MICs were performed using the broth microdilution methodology following the NCCLS procedure (NCCLS Document M7-A4 Vol. 17 No. 2 January 1997) in presence of 0.02% albumine bovine serum with inocula of approximately 5×10⁵ cfu/mL. The media employed included cation-adjusted Mueller-Hinton (MH) broth (Difco Laboratories, Detroit, Mich., USA) supplemented or not with 30% (v/v) bovine serum. Tests were read after 24 h incubation at 25-37° C. The antibiotic activity of 97518 and its derivatives 1-13 are reported in Table 6.

TABLE 6
antimicrobial activity of 97518 and its derivatives 1-13

| | | Minimal Inhibitory Concentrations (μg/mL) | | | | | | | | | | | | | |
|---|---|---|---|---|---|---|---|---|---|---|---|---|---|---|---|
| microorganism | code | 97518 | 1 | 2 | 3 | 4 | 5 | 6 | 7 | 8 | 9 | 10 | 11 | 12 | 13 |
| Staphylococcus aureus | 3797 | >128 | 4 | 16 | 1 | 0.50 | 8 | 2 | 1 | 2 | 32 | 8 | 8 | 2 | 16 |
| VISA Met-R | | | | | | | | | | | | | | | |
| Staphylococcus aureus | 3798 | >128 | 2 | 4 | 0.50 | 0.50 | 2 | 1 | 0.50 | 1 | 32 | 4 | 4 | 1 | 16 |
| VISA | | | | | | | | | | | | | | | |
| S. pyogenes | 49 | 0.25 | na | na | 2 | 0.50 | na | na | na | na | na | na | na | 0.50 | 0.25 |
| S. epidermidis ATCC12228 | 147 | 64 | 2 | 8 | 2 | 2 | 4 | 2 | 1 | 1 | 16 | 8 | 8 | 1 | 16 |
| S. haemolyticus Met-R | 1729 | 128 | 32 | 32 | 1 | 0.50 | 32 | >128 | 0.25 | 1 | 16 | 16 | 32 | 1 | 128 |
| S. haemolyticus Met-S | 1730 | 128 | 4 | 8 | <0.125 | <0.125 | 4 | 2 | <0.125 | 1 | 16 | 4 | 4 | <0.125 | 64 |
| Enterococcus faecium | 568 | >128 | 2 | 16 | 2 | 1 | 8 | 2 | 1 | 2 | 8 | 8 | 16 | 2 | 16 |
| VanS | | | | | | | | | | | | | | | |
| E. faecalisVanS | 559 | 128 | 1 | 16 | 4 | 4 | 8 | 2 | 4 | 1 | 16 | 16 | 16 | 8 | 16 |
| E. faecalisVanA | 560 | 128 | 1 | 16 | 4 | 2 | 8 | 2 | 2 | 1 | 16 | 16 | 16 | 4 | 32 |
| E. faecium VanA | 569 | >128 | 2 | 16 | 1 | 1 | 8 | 1 | 1 | 2 | 16 | 8 | 8 | 1 | 16 |
| Escherichia coli | 47 | >128 | >128 | >128 | >128 | >128 | >128 | >128 | >128 | 2 | >128 | >128 | >128 | >128 | >128 |
| C. albicans ATCC 90028 | 145 | 64 | 64 | 64 | 64 | 64 | 32 | 32 | 64 | 0.25 | 32 | 64 | 64 | 64 | 64 |

1. A carboxyamide derivative having the following formula II 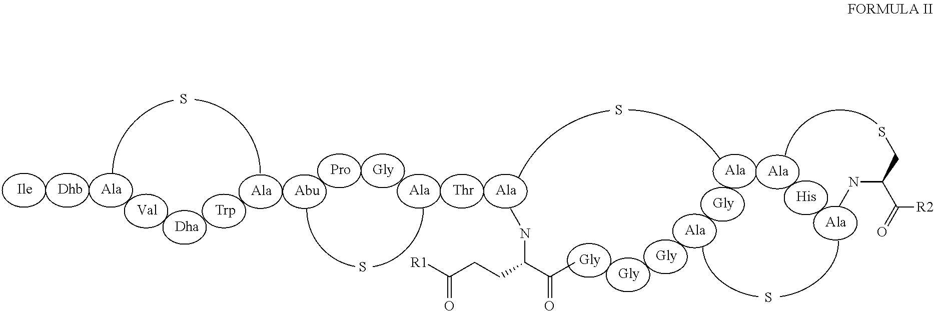
wherein one of R₁ and R₂ is —NR₃R₄, and the other is selected from the group consisting of —OH and —NR₃R₄, and wherein R₃ and R₄ are independently selected from the group consisting of:
(a) hydrogen;
(b) an alkyl of 1 to 20 carbon atoms;
(c) an alkenyl of 2 to 20 carbon atoms;
(d) an alkynyl of 2 to 20 carbon atoms;
(e) a cycloalkyl of 3 to 8 carbon atom optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms;
(f) a phenyl radical optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms;
(g) a benzyl radical optionally substituted on the phenyl ring by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms;
(h) a naphthyl radical optionally substituted by one or two substituents selected from halo, lower alkyl of 1 to 4 carbon atoms optionally substituted by 1 to 3 halogen atoms, and lower alkoxy of 1 to 4 carbon atoms optionally substituted by 1 to 3 halogen atoms;
(i) a group of formula
—(CH₂)ₙOR₅
in which n represents an integer from 2 to 8 and R₅ represent
(1) hydrogen;
(2) (C₁-C₄)alkyl;
(3) a cycloalkyl of 3 to 8 carbon atom optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms;
(4) a phenyl radical optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms; and
(j) a group of formula
—(CH₂)ₙNR₆R₇
in which n represents an integer from 2 to 8 and R₆ and R₇ independently represent
(1) hydrogen;
(2) (C₁-C₄)alkyl;
(3) a cycloalkyl of 3 to 8 carbon atom optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms;
(4) phenyl radical optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms;
(5) a benzyl radical optionally substituted on the phenyl ring by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms
(6) R₆ and R₇ taken together represent a —(CH₂)₃, —(CH₂)₄—, —(CH₂)₂—O—(CH₂)₂, —(CH₂)₂—S—(CH₂)₂; or
(7) R₆ and R₇ taken together with the adjacent nitrogen atom represent: a piperazine moiety which may be substituted in position 4 with a substituent selected from (C₁-C₄)alkyl, (C₃-C₈)cycloalkyl, pyridyl, benzyl and substituted benzyl wherein the phenyl moiety bears 1 or 2 substituents selected from chloro, bromo, nitro, (C₁-C₄)alkyl and (C₁-C₄)alkoxy.

2. A carboxyamide derivative according to claim 1, wherein:
one of R₁ and R₂ is —NR₃R₄, and the other is selected from the group consisting of —OH and —NR₃R₄, and wherein R₃ and R₄ are independently selected from the group consisting of:
(a) an alkyl of 1 to 12 carbon atoms;
(b) an alkenyl of 3 to 10 carbon atoms;
(c) a cycloalkyl of 5 to 6 carbon atom optionally substituted by one or two substituents independently selected from lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, lower alkyl of 1 to 4 carbon atoms, and lower alkoxy of 1 to 4 carbon atoms;
(d) a phenyl radical optionally substituted by one or two substituents independently selected from halo, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms;
(e) a benzyl radical optionally substituted on the phenyl ring by one or two substituents independently selected from halo, cyano, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms;
(f) a naphthyl radical optionally substituted by one or two substituents selected from halo, lower alkyl of 1 to 4 carbon atoms, and lower alkoxy of 1 to 4 carbon atoms;
(g) a group of formula
—(CH₂)ₙOR₅
in which n represents an integer 2 to 5 and R₅ represent
(1) hydrogen;
(2) (C₁-C₄)alkyl;
(3) a cycloalkyl of 5 to 6 carbon atom optionally substituted by one or two substituents independently selected from halo, cyano, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms;
(4) a phenyl radical optionally substituted by one or two substituents independently selected from halo, cyano, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms;
(h) a group of formula
—(CH₂)ₙNR₆R₇
in which n represents an integer from 2 to 8 and R₆ and R₇ independently represent
(1) hydrogen;
(2) (C₁-C₄)alkyl;
(3) a cycloalkyl of 3 to 6 carbon atom optionally substituted by one or two substituents independently selected from halo, cyano, lower alkyl of 1 to 4 carbon atoms, lower alkoxy of 1 to 4 carbon, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon, and lower alkoxy of 1 to 4 carbon atoms;
(4) a phenyl radical optionally substituted by one or two substituents independently selected from halo, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms;
(5) a benzyl radical optionally substituted on the phenyl ring by one or two substituents independently selected from halo, cyano, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms;
(6) R₆ and R₇ taken together represent a —(CH₂)₃, —(CH₂)₄—, —(CH₂)₂—O—(CH₂)₂, —(CH₂)₂—S—(CH₂)₂; or
(7) R₆ and R₇ taken together with the adjacent nitrogen atom represent: a piperazine moiety which may be substituted in position 4 with a substituent selected from (C₁-C₄)alkyl, (C₃-C₈)cycloalkyl, pyridyl, benzyl and substituted benzyl wherein the phenyl moiety bears 1 or 2 substituents selected from chloro, bromo, nitro, (C₁-C₄)alkyl and (C₁-C₄)alkoxy.

3. A carboxyamide derivative according to claim 1, wherein:
one of R₁ and R₂ is —NR₃R₄, and the other is selected from the group consisting of —OH and —NR₃R₄, and wherein R₃ and R₄ independently have the formula
—(CH₂)ₙNR₆R₇
in which n represents an integer from 2 to 8 and R₆ and R₇ are independently selected from the group consisting of:
(a) hydrogen;
(b) (C₁-C₄)alkyl; or
(c) a cycloalkyl of 3 to 6 carbon atom optionally substituted by one or two substituents independently selected from halo, cyano, lower alkyl of 1 to 4 carbon atoms, lower alkoxy of 1 to 4 carbon, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon, and lower alkoxy of 1 to 4 carbon atoms;
(d) a phenyl radical optionally substituted by one or two substituents independently selected from halo, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms;
(e) a benzyl radical optionally substituted on the phenyl ring by one or two substituents independently selected from halo. cyano, lower alkyl of 1 to 4 carbon atoms, lower alkoxy of 1 to 4 carbon atoms, phenyl, phenyl-lower alkyl of 1 to 4 carbon atoms, phenoxy, phenoxy-lower alkyl of 1 to 4 carbon atoms;
(f) R₆ and R₇ taken together represent a —(CH₂)₃, —(CH₂)₄—, —(CH₂)₂—O—(CH₂)₂, —(CH₂)₂—S—(CH₂)₂; or
(g) R₆ and R₇ taken together with the adjacent nitrogen atom represent: a piperazine moiety which may be substituted in position 4 with a substituent selected from (C₁-C₄)alkyl, (C₃-C₈)cycloalkyl, pyridyl, benzyl and substituted benzyl wherein the phenyl moiety bears 1 or 2 substituents selected from chloro, bromo, nitro, (C₁-C₄)alkyl and (C₁-C₄)alkoxy.

4. A carboxyamide derivative according to claim 1, wherein:
one of R₁ and R₂ is —NR₃R₄, and the other is selected from the group consisting of —OH and —NR₃R₄, and wherein R₃ and R₄ are independently
—(CH₂)ₙNR₆R₇
in which n represents an integer from 2 to 4 and R₆ and R₇ independently represent
hydrogen or (C₁-C₄)alkyl.

5. A carboxyamide derivative according to claim 1, wherein at least one of said —NR₃R₄ has the following formula:
—NH—(CH₂)₂—NH₂; —NH(CH₂)₃NH₂—NH—(CH₂)₄—NH₂; —NH(CH₂)₃NHCH₃—NH—(CH₂)₃—N(CH₃)₂; —NH—(CH₂)₃N(C₂H₅)₂—NH—(CH₂)₃N(C₃H₇)₂; —NH—(CH₂)₃N(C₄H₉)₂—NH—(CH₂)₅N(CH₃)₂; —NH(CH₂)₆N(CH₃)₇—NH(CH₂)₆NHCH₃; —N[(CH₂)₂NH₂]₂—N[(CH₂)₃NH₂]₂; —N[(CH₂)₂N(CH₃)₂]₂—N[(CH₂)₃N(CH₃)₂]₂; —N[(CH₂)₄NH₂]₂

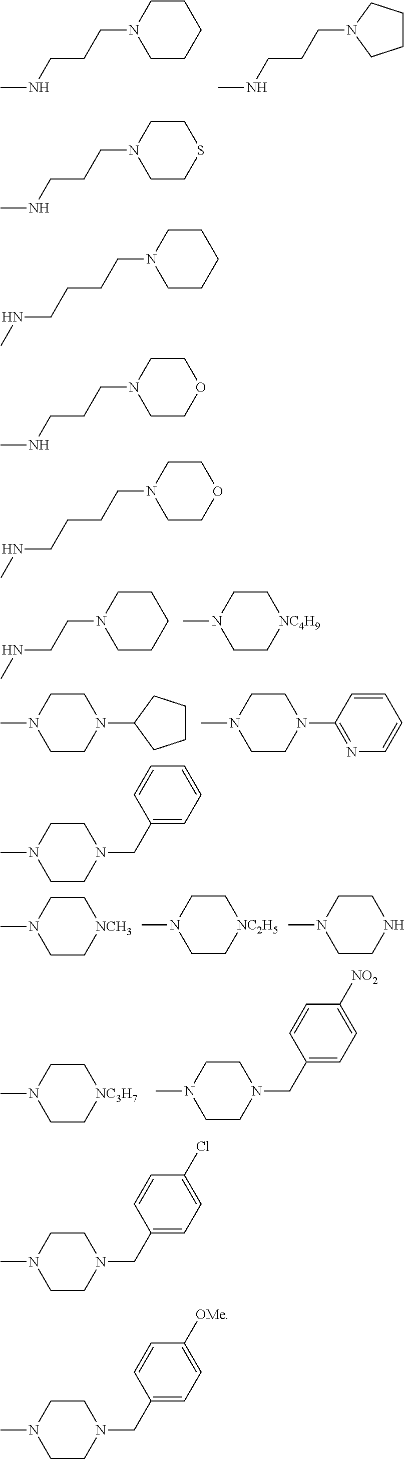

6. A carboxyamide derivative according to claim 1, selected from the group consisting of:

| Compound | R₁ | R₂ |
|---|---|---|
| 1 | 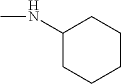 | 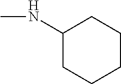 |
| 2 | 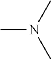 | 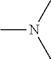 |
| 3 | 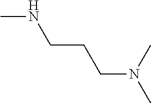 | 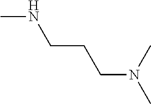 |
| 4 | 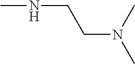 | 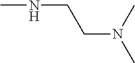 |
| 5 | 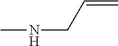 | 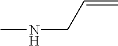 |
| 6 | 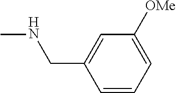 | 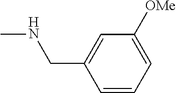 |
| 7 | 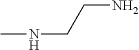 | 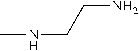 |
| 8 | 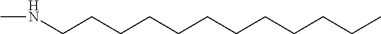 | 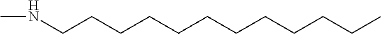 |
| 9 | 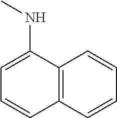 | 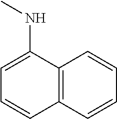 |
| 10 | 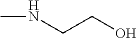 | 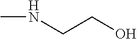 |
| 11 | 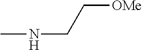 | 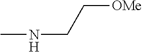 |
| 12 | 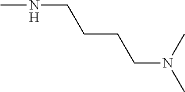 | 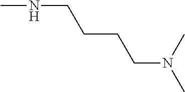 |
| 13 | 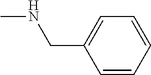 | 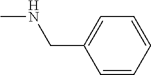 |
| 14 | 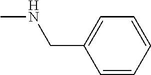 | OH |
| 15 | OH | 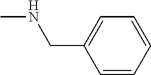 |
| 16 | 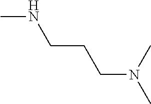 | OH |
| 17 | OH | 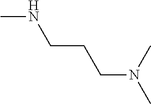 |
| 18 | 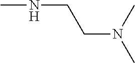 | OH |
| 19 | OH | 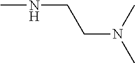 |
| 20 | 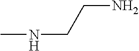 | OH |
| 21 | OH | 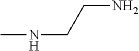 |
| 22 | 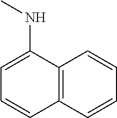 | OH |
| 23 | OH | 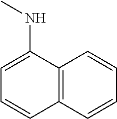 |
| 24 | 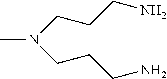 | 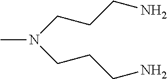 |
| 25 | 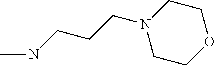 | 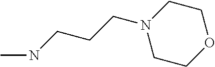 |
| 26 | 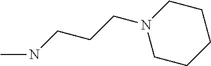 | 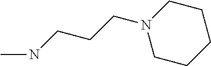 |
| 27 | 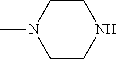 | 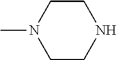 |
| 28 | 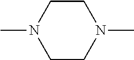 | 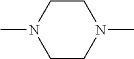 |
| 29 | 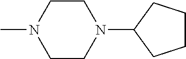 | 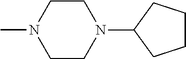 |
| 30 | 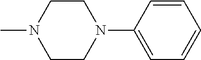 | 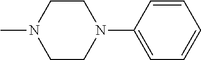 |
| 31 | 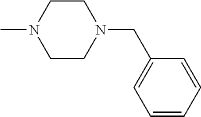 | 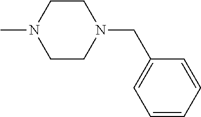 |

7. A carboxyamide derivative according to claim 6, selected from the group consisting of:

| Com- | | |
|---|---|---|
| pound | R₁ | R₂ |
| 3 | 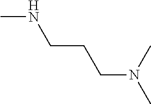 | 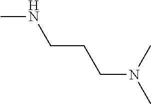 |
| 4 | 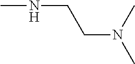 | 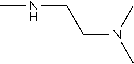 |
| 7 | 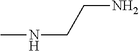 | 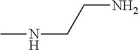 |
| 12 | 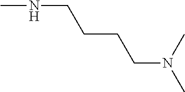 | 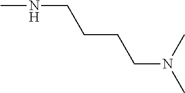 |

8. A method for the preparation of a carboxyamide derivative according to claim 1, comprising the condensation reaction between a starting compound selected from the group consisting of formula (I) and formula (III) 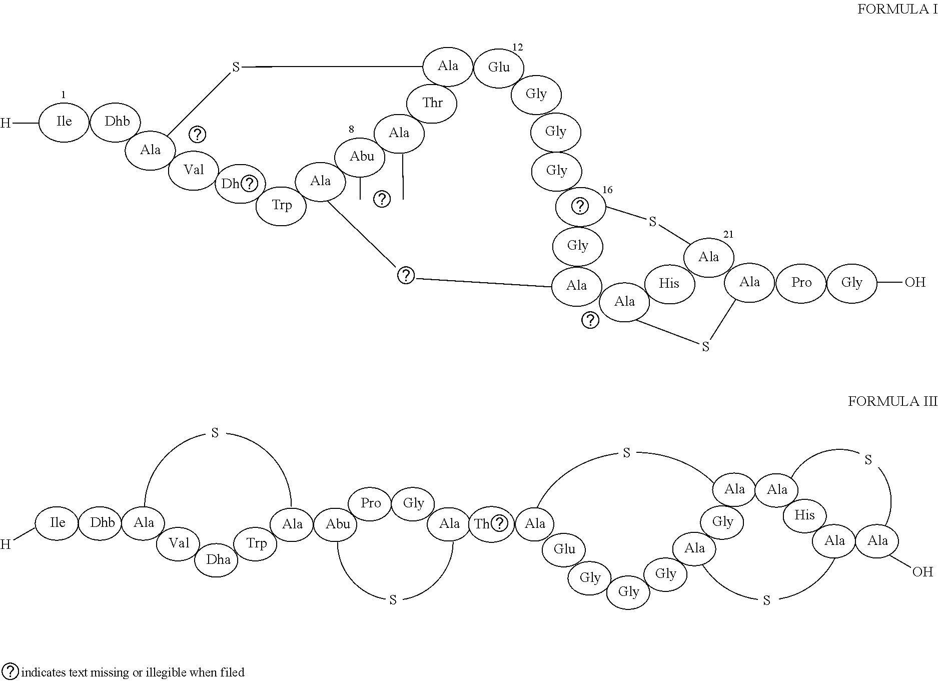
and an amine of general formula HNR₃R₄, wherein R₃ and R₄ are independently selected from the group consisting of:
(a) hydrogen;
(b) an alkyl of 1 to 20 carbon atoms;
(c) an alkenyl of 2 to 20 carbon atoms;
(d) an alkynyl of 2 to 20 carbon atoms;
(e) a cycloalkyl of 3 to 8 carbon atom optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms;
(f) a phenyl radical optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms;
(g) a benzyl radical optionally substituted on the phenyl ring by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms;
(h) a naphthyl radical optionally substituted by one or two substituents selected from halo, lower alkyl of 1 to 4 carbon atoms optionally substituted by 1 to 3 halogen atoms, and lower alkoxy of 1 to 4 carbon atoms optionally substituted by 1 to 3 halogen atoms;
(i) a group of formula
—(CH₂)ₙOR₅
in which n represents an integer from 2 to 8 and R₅ represent
(1) hydrogen;
(2) (C₁-C₄)alkyl;
(3) a cycloalkyl of 3 to 8 carbon atom optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms;
(4) a phenyl radical optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms;
(j) a group of formula
—(CH₂)ₙNR₆R₇
in which n represents an integer from 2 to 8 and R₆ and R₇ independently represent
(1) hydrogen;
(2) (C₁-C₄)alkyl;
(3) a cycloalkyl of 3 to 8 carbon atom optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms;
(4) phenyl radical optionally substituted by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms;
(5) a benzyl radical optionally substituted on the phenyl ring by one or two substituents independently selected from halo, cyano, lower alkyl of 1 to 4 carbon atoms optionally substituted by 1 to 3 halogen atoms, lower alkoxy of 1 to 4 carbon atoms optionally substituted by 1 to 3 halogen atoms, phenyl, phenyl-lower alkyl of 1 to 4 carbon atoms, phenoxy, phenoxy-lower alkyl of 1 to 4 carbon atoms wherein the phenyl and the phenyl portion of the phenyl lower-alkyl, phenoxy and phenoxy-lower alkyl group is optionally substituted by one or two substituents selected from halo, cyano, lower alkyl of 1 to 4 carbon atoms optionally substituted by 1 to 3 halogen atoms, and lower alkoxy of 1 to 4 carbon atoms optionally substituted by 1 to 3 halogen atoms;
(6) R₆ and R₇ taken together represent a —(CH₂)₃, —(CH₂)₄—, —(CH₂)₂—O—(CH₂)₂, —(CH₂)₂—S—(CH₂)₂;
(7) R₆ and R₇ taken together with the adjacent nitrogen atom represent: a piperazine moiety which may be substituted in position 4 with a substituent selected from (C₁-C₄)alkyl, (C₃-C₈)cycloalkyl, pyridyl, benzyl and substituted benzyl wherein the phenyl moiety bears 1 or 2 substituents selected from chloro, bromo, nitro, (C₁-C₄)alkyl and (C₁-C₄)alkoxy;
in the presence of a condensing agent.

9. The method according to claim 8, wherein said R₃ and R₄ are independently selected from the group consisting of:
—NH—(CH₂)₂—NH₂; —NH(CH₂)₃NH₂—NH—(CH₂)₄—NH₂; —NH(CH₂)₃NHCH₃—NH—(CH₂)₃—N(CH₃)₂; —NH—(CH₂)₃N(C₂H₅)₂—NH—(CH₂)₃N(C₃H₇)₂; —NH—(CH₂)₃N(C₄H₉)₂—NH—(CH₂)₅N(CH₃)₂; —NH(CH₂)₆N(CH₃)₇—NH(CH₂)₆NHCH₃; —N[(CH₂)₂NH₂]₂—N[(CH₂)₃NH₂]₂; —N[(CH₂)₂N(CH₃)₂]₂—N[(CH₂)₃N(CH₃)₇]₇; —N[(CH₂)₄NH₂]₂

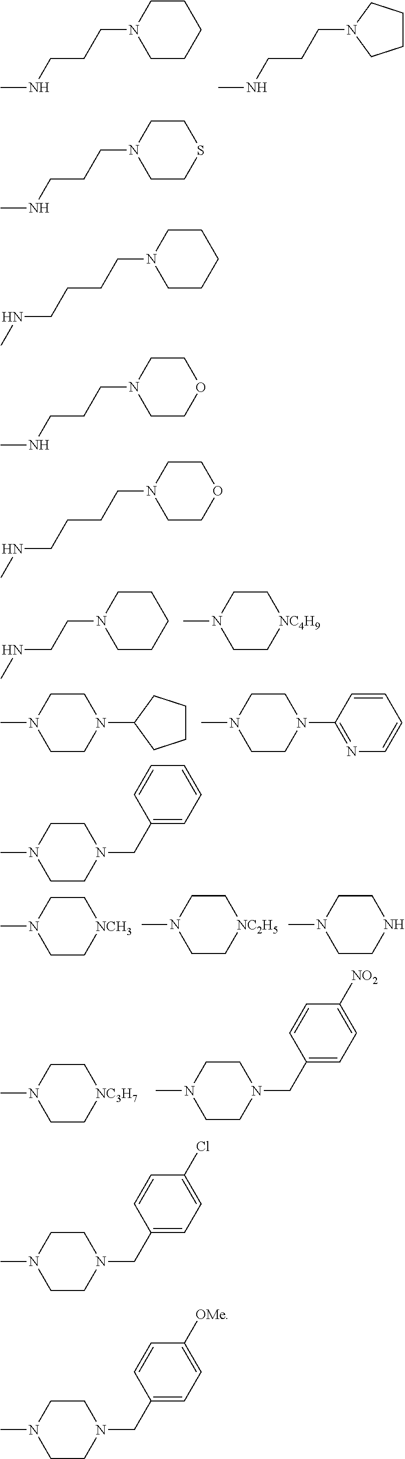

10. The method according to claim 8 wherein said condensation reaction is carried out in the presence of at least a condensing agent and at least a solvent selected from the group consisting of: organic amides, ethers of glycols and polyols, phosphoramide derivatives, sulfoxides dimethylformamide, dimethoxyethane, hexamethyl phosphoroamide, dimethylsulphoxide, dioxane, N-methylpyrrolidone and mixtures thereof.

11. The method according to claim 8, wherein said condensation reaction is carried out at a temperature ranging from 0° C. to 50° C.

12. A pharmaceutical composition comprising a carboxyamide derivative according to claim 1, or a pharmaceutically acceptable salt thereof.

13. The pharmaceutical composition according to claim 12 comprising a pharmaceutically acceptable carrier.

14. The pharmaceutical composition according to claim 12 characterized in that it is orally, topically or parenterally administrable.

15. The pharmaceutical composition according to claim 12 characterized in that it is in a form selected from the group consisting of capsules, tablets, liquid solutions, aqueous suspensions, oily solutions or suspensions, hydrophobic or hydrophilic bases as ointments, creams, lotions, paints, or powders.

16. A method of treating a bacterial infection in a mammal, the method comprising the administration of the composition of claim 12 or an acceptable salt thereof to a subject in need thereof.

17. A method of treating or preventing a bacterial infection in a mammal, the method comprising the administration of the composition of claim 12 or a pharmaceutically acceptable salt, formulation, or composition thereof to a subject in need thereof.

18. The method of claim 16, wherein said bacterial infection is caused by at least one bacteria from the group consisting of enterococci, streptococci and staphylococci.

19. The method of claim 16, wherein said bacterial infection is caused by at least one bacteria from the group consisting of Clostridium difficile, Staphylococcus spp., Streptococcus spp, Enterococcus spp, Propionibacterium acnes, and Moraxella spp.

20. The method of claim 16, wherein the dosage range is between 1 and 40 mg of active ingredient per Kg of body weight.
